(19) United States
(12) Patent Application Publication
(10) Pub. No.: US 2004/0072284 A1
(43) Pub. Date: Apr. 15, 2004

(54) Critical association concentration assembly

(76) Inventors: Kevin J. McCarthy, Shreveport, LA (US); Mark E. Lauer, Olmsted Falls, OH (US); Ryan J. Bienvenu, Shreveport, LA (US); Benjamin S. Scruggs, Shreveport, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/272,583

(22) Filed: Oct. 15, 2002

(51) Int. Cl. C12P 21/02; C12P 19/04; C12P 7/62; C12N 5/06

(52) U.S. Cl. 435/69.1; 435/101; 435/135; 435/325

(57) ABSTRACT

An apparatus and method for inducing rapid, in vitro growth and development of robust basal lamina matrix that is similar to the in vivo matrix has been developed. The apparatus is a critical assembly concentration apparatus that limits the diffusion of secreted basal membrane components from mammalian cells to the immediate pericellular environment, while maintaining a nutrient rich environment for the cells to thrive. The CAC comprises a culture medium holder and a semipermeable membrane that limits diffusion based on size of molecules. The semipermeable membrane is chosen to inhibit protein diffusion, but to allow smaller nutrients to diffuse freely. The CAC apparatus was tested with a tumor cell line, L-2 cells, and was found to decrease the time necessary to assemble a robust matrix to about 24 to about 72 hr. The CAC design was modified to bio-engineer a three-dimensional matrix using smooth muscle cells. The three-dimensional design can be used to grow artificial tissues or organs, e.g., an artificial vascular wall.

This invention pertains to an apparatus and a method to enhance natural cell growth in cell culture, particularly the rapid growth of pericellular basement membrane matrices.

Basement membranes (“BM”) are specialized extracellular matrices that separate epithelial cells from the underlying stroma and surrounding smooth muscle, nerve and fat cells. See R. Vracko, “Basal Lamina Scaffold-anatomy and Significance for Maintenance of Orderly Tissue Structure,” Am. J. Pathol., vol. 77, pp. 314-346 (1974). The basal lamina (“BL”), a component of the BM, provides structural support and tissue compartmentalization and serves as a selective barrier. Early studies on BL and BM were limited to morphological and histochemical methods. Biochemical characterization of BLs and BMs and their constituent macromolecules were complicated by the relative paucity and insolubility of BM components under normal physiological conditions. The discovery of the Englbreth-Holm-Swarm (“EHS”) sarcoma, a transplantable tumor with extracellular matrices that are primarily BL macromolecules, enabled large quantities of BL components to be obtained. See R. W. Orkin et al., “A Murine Tumor Producing a Matrix of Basement Membrane,” J. Exp. Med., vol. 145, pp. 204-220 (1977); and R. L. Swarm, “Transplantation of a Murine Chondrosarcoma in Mice of Different Inbred Strains,” J. Natl. Cancer Inst., vol. 31, p. 953 (1963). The subsequent isolation of tumor cell lines, e.g., L-2 cells (rat) and PYS cells (mouse), that secrete large amounts of matrix material in a soluble form, also facilitated the isolation of individual extracellular matrix (“ECM”) components. See H. J. Merker et al., “The Morphology of Basement Membrane Formation,” Eur. J. Cell. Biol., vol. 26, pp.111-120 (1981); U. M. Wewer, “Heparan Sulfate Proteoglycans Made by Different Basement-membrane-producing Tumors Have Immunological and Structural Similarities,” Differentiation, vol. 30, pp. 61-67 (1985); and B. L. Hogan et al., “Synthesis and Localization of Two Sulphated Glycoproteins Associated with Basement Membranes and the Extracellular Matrix,” J. Cell. Biol., vol. 95, pp. 197-204 (1982).

Although early immunohistochemical studies with polyclonal antiserum directed against BM proteins suggested that all BMs were composed of identical proteins, later work indicated that the constituent matrix molecules of BMs are often tissue-specific. These tissue-specific differences are found in the type IV collagen chains, laminin isoforms, and proteoglycan components. See G. R. Martin et al., “The Regulation of Basement Membrane Formation and Cell-matrix Interactions by Defined Supramolecular Complexes,” Ciba Found. Symp., vol. 108, pp. 197-212 (1984); K. J. McCarthy et al., “Basement Membrane Chondroitin Sulfate Proteoglycans: Localization in Adult Rat Tissues,” J. Histochem. Cytochem., vol. 38, pp.1479-1486 (1990); J. R. Sanes et al., “Molecular Heterogeneity of Basal Laminae: Isoforms of Laminin and Type IV Collagen at the Neuromuscular Junction and Elsewhere,” J. Cell Biol., vol. 111, pp.1685-1699 (1990); J. H. Miner et al., “Collagen IV Alpha 3, Alpha 4, and Alpha 5 Chains in Rodent Basal Laminae: Sequence, Distribution, Association with Laminins, and Developmental Switches,” J. Cell Biol., vol. 127, pp. 879-891 (1994); J. R. Miner et al., “Molecular Cloning of a Novel Laminin Chain, Alpha 5, and Widespread Expression in Adult Mouse Tissues,” J. Biol. Chem., vol. 270, pp. 28523-28526 (1995); and A. J. Groffen et al., “Agrin Is a Major Heparan Sulfate Proteoglycan in the Glomerular Basement Membrane,” J. Histochem. Cytochem., vol. 46, pp. 19-27 (1998).

Studies have shown that BMs dramatically influence the behavior of cells, especially during regeneration. BM serves as a scaffold along which regenerating cells migrate, and helps spatially organize various growth factors and other proteins important for regeneration. The interaction of growth factors with BM proteins can serve either to enhance the activity of the growth factors by maintaining a close proximity with growth-factor surface receptors, or to decrease their activity by preventing access to the surface receptors. BM proteins may also activate specific cell-Express adhesion surface receptors to influence gene expression, cellular differentiation, and protein production.

Although BMs have been extensively studied in in vivo animal models, in vitro modeling of BMs in certain cell types is difficult. While some cells, e.g., epithelial cells, readily produce a BM in culture, other cells produce an in vitro BM only after prolonged culture, or require co-culture with nurse cells that can complicate in vitro modeling. See P. Delvoye et al., “Fibroblasts Induce the Assembly of the Macromolecules of the Basement Membrane,” J. Invest. Dermatol, vol.90, pp. 276-282 (1988); and R. H. Tammi et al., “A Preformed Basal Lamina Alters the Metabolism and Distribution of Hyaluronan in Epidermal Keratinocyte Cultures Grown on Collagen Matrices,” Histochem. Cell Biol., vol. 113, pp. 265-277 (2000). In addition to the production of a native BM by cells, cell-matrix interactions can be studied using a commercially available “generic” pericellular matrix, such as Matrigel® (Becton Dickinson, Bedford, Mass.). Although the Matrigel® matrix contains BM proteins that facilitate cell growth for many tissues, it is known that the basement membrane proteins and proteoglycans secreted by the natural tissue cells differ in composition from those present in the Matrigel® matrix.

L-2 cells are a rat tumor cell line derived from the parietal endoderm cells that produce Reichert's membrane, abasement membrane transiently present during rodent development. See L. Kulay et al., “A Histochemical Study on the Reichert Membrane of the Rat's Placenta,” Acta. Histochem., vol. 22, pp. 309-321 (1965). The term “basal lamina” describes the pericellular matrices assembled by L-2 cells, a term often used interchangeably with “basement membrane.” In the strictest sense, however, the term “basement membrane” refers to the complete extracellular matrix that underlies the epithelia, and that has as constituent parts the basal lamina and the reticular lamina. See E. D. Hay, Cell Biology of Extracellular Matrix (2^{nd} ed., New York and London, Plenum Press, 1991). Because L-2 cells primarily secrete components of the basal lamina, the pericellular matrix assembled by these cells has a composition more like a basal lamina. In vivo, the native parietal endoderm cells assemble a thick BL over a period of several days.

Although L-2 cells secrete all the major components of BL, the components fail to assemble a significant matrix in culture under routine conditions. P. Delvoye et al., 1988, discloses the use of a model system that induces L-2 cells to assemble a basal lamina when grown in the presence of fibroblasts. Although suggesting the possibility that a precise concentration of BM components diffusing from L-2 cells may be necessary to assemble BL matrices, this possibility was found unlikely because a higher laminin concentration did not influence the assembly of BL matrices. In pure cultures of L-2 cells, the basement membrane components were aggregated in a granular pattern. The researchers concluded that one or more molecules synthesized by fibroblasts were responsible for triggering the assembly of the major components of basement membranes into a matrix network.

An unfilled need exists for a new apparatus and method to create a cell culture environment that enhances the growth of an extracellular matrix that better resembles the in vivo composition.

We have discovered an apparatus and a method for inducing rapid in vitro growth and development of a robust basal lamina matrix similar in composition to the in vivo matrix. The device is named a “Critical Assembly Concentration Apparatus” (“CAC”). The CAC limits the diffusion of secreted basal membrane components while maintaining a nutrient-rich environment. The CAC comprises a culture medium holder and a semipermeable membrane that limits diffusion based on size of molecules. The semipermeable membrane is chosen to inhibit protein diffusion, but to allow smaller nutrients to diffuse freely. The CAC apparatus was tested with a tumor cell line, L-2 cells, and was found to decrease the time necessary to assemble a robust matrix to about 24 to about 72 hr. The CAC design was modified to bio-engineer a three-dimensional matrix using smooth muscle cells. The three-dimensional design can be used to grow artificial tissues or organs, e.g., an artificial vascular wall.

### BRIEF DESCRIPTION OF DRAWINGS

FIG. 1 is a cross-sectional schematic diagram of one embodiment of the two-dimensional CAC apparatus, illustrating a monolayer of cells with the limiting membrane inside a culture holder.

FIG. 2A is a perspective schematic diagram of part of one embodiment of the three-dimensional CAC apparatus illustrating the central core and limiting membrane, but not the culture holder.

FIG. 2B is a cross-sectional schematic diagram of one embodiment of the three-dimensional CAC apparatus, illustrating the central core, a layer of cells, a limiting membrane, and the culture holder.

FIG. 3A is a side schematic diagram of part of the perfusion embodiment of the three-dimensional CAC apparatus, illustrating the hollow center core with perfusion channels cut in the sides.

FIG. 3B is a cross-sectional schematic diagram of the perfusion embodiment of the three-dimensional CAC apparatus, illustrating the central core with channels, a limiting membrane around the core, a layer of cells, another limiting membrane, and the culture holder.

This invention provides a reliable, inexpensive apparatus and method for effectively limiting the diffusion of proteins secreted from mammalian cells (e.g., BM glycoproteins and proteoglycans) to the immediate pericellular environment. The basic design comprises a culture medium holder and a semipermeable membrane chosen to promote the free exchange of growth-factors and nutrients from the culture medium to the cells, but to retard the movement of secreted proteins away from the cells.

The novel apparatus, named a “Critical Assembly Concentration Apparatus” (“CAC”), has several advantages over current culture techniques. First, the CAC promotes the rapid development of true native basal lamina matrices, matrices with a composition the same as or similar to that which would be seen in vivo. The CAC works particularly well with cells that appear to secrete extracellular matrix (“ECM”) proteins from their entire cell surfaces in a random direction, such as tumor cells (e.g., L-2 cells), smooth muscle cells, neurons, endothelial cells, fibroblasts and mesenchymal stem cells. Second, once the cells have developed the matrix in the CAC, the matrix or the cells are readily accessible for subsequent cell culture studies. Third, the CAC promotes the development of a basal lamina matrix by cell lines that otherwise will not readily assemble a matrix in vitro. Fourth, the CAC design can be used in any culture situation where limiting the diffusion of cellular secreted products would be beneficial, such as studying the effect of secreted growth factors, chemokines, or other cell proteins. Finally, the CAC design is easily modified to grow matrices in three dimensions. Three-dimensional matrices are important in bioengineering artificial tissues and organs, e.g., an artificial vascular wall.

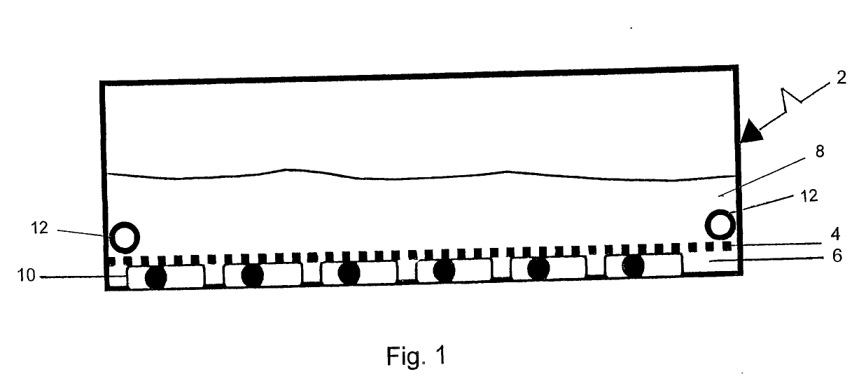

FIG. 1 is a schematic diagram of one embodiment of a two-dimensional CAC apparatus comprising a culture medium holder 2 having a culture well, and a limiting membrane 4. The culture medium holder may have multiple wells (e.g., 1-well, 8-well, or 24-well), and may be made from any impermeable and biologically compatible material, e.g., stainless steel, glass, or polymers such as polypropylene.

The limiting membrane 4 comprises a removable semipermeable membrane which divides the culture medium holder into a first chamber 8 and a second chamber 6. In this embodiment, a layer of cells 10 is plated in a small volume of culture medium in the second chamber 6, and then the limiting membrane 4 is placed on top. See FIG. 1. Then additional culture medium is added to the first chamber 8. The dimensions and shape of the removable, semipermeable limiting membrane 4 are adjusted to fit the shape and size of the culture medium holder 2.

The distance between the semipermeable limiting membrane 4 and the layer of cells 10 can be adjusted to restrict the diffusion of molecules secreted by the cells (e.g., glycoproteins and proteoglycans) to the adjacent pericellular area. Without wishing to be bound by this theory, it is believed that the optimal distance is less than about 1 mm, preferably between about 50 nm to about 1000 nm. This distance can be adjusted to optimize the growth of the matrix; in general, the smaller the distance, the faster the growth. A stabilizer 12 such as a silicon rubber O-ring (Small Parts, Inc., Miami Lakes, Fla.), is preferably placed on top of the limiting membrane 4 to secure the membrane 4 in the bottom of culture medium holder 2. Optionally, a second stabilizer (not shown) may be placed below the limiting membrane 4 to support the limiting membrane 4 and set the distance between the limiting membrane 4 and the cells. The pore exclusion size of the limiting membrane 4 is chosen to allow a free exchange of growth factors and nutrients in the culture medium from first chamber 8 into second chamber 6, while restricting the flow of proteins out of chamber 6. For most culturing situations, the limiting membrane 4 will be chosen to prevent diffusion of any molecule with a molecular weight greater than about 100,000 Daltons. The chosen cut-off is determined by the size of the smallest secreted molecule to retain in chamber 6. In culturing basement membranes, the smallest secreted molecule known to be of interest is entactin (150,000 Daltons). The pore size of the limiting membrane can be any size below the cut-off value that allows diffusion of the nutrients and growth factors supplied in the culture medium. In the prototype, the limiting membrane 4 had a molecular weight cut-off of 12,000 to 14,000 Daltons. Conceivably, the pore size could be as low as about 3,500 Daltons.

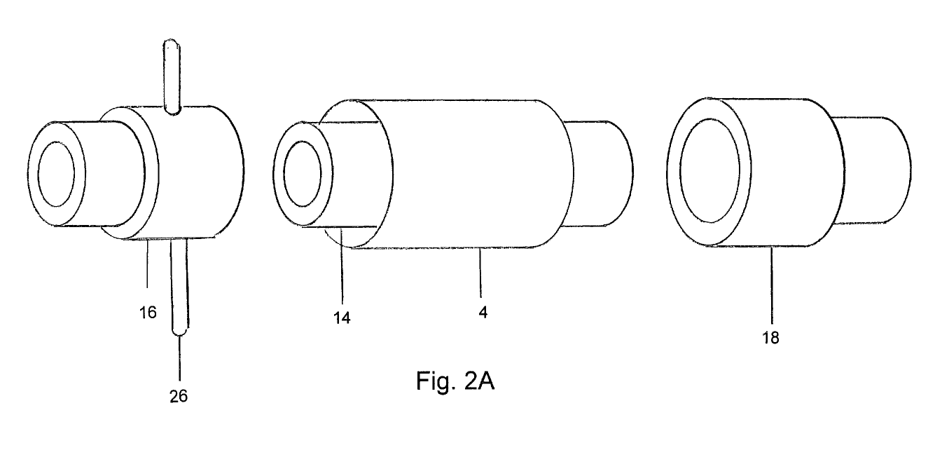

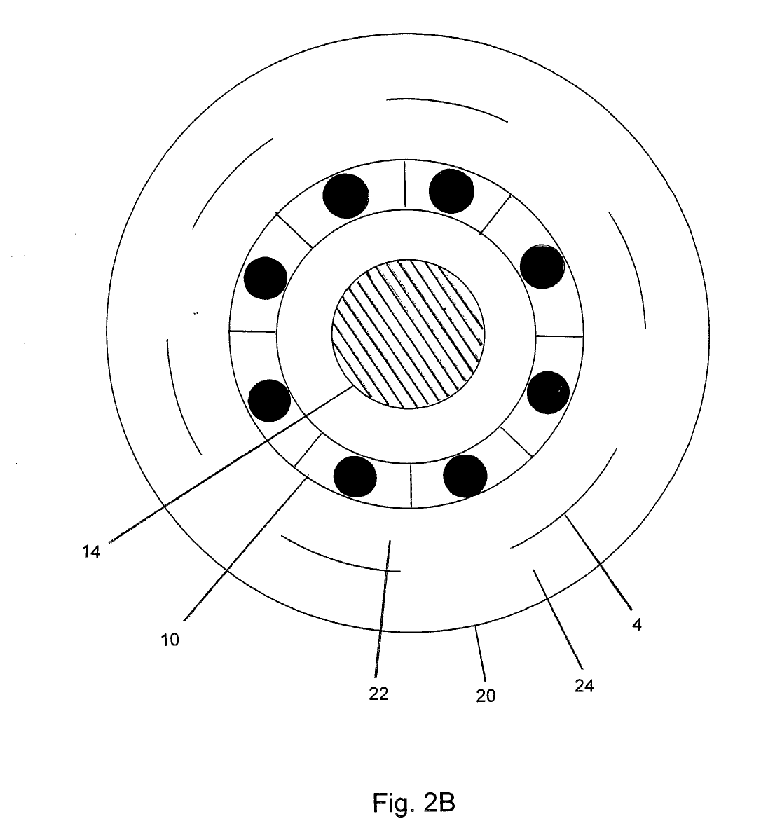

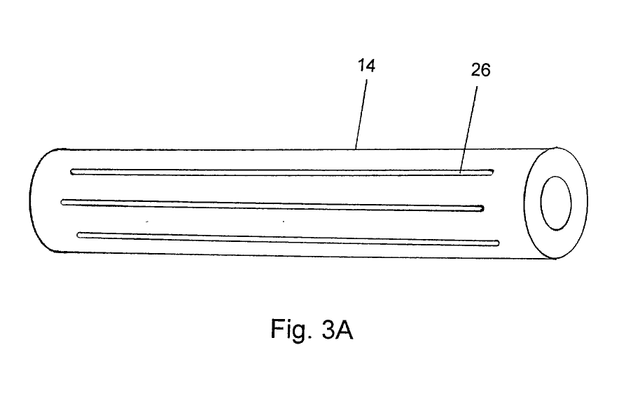

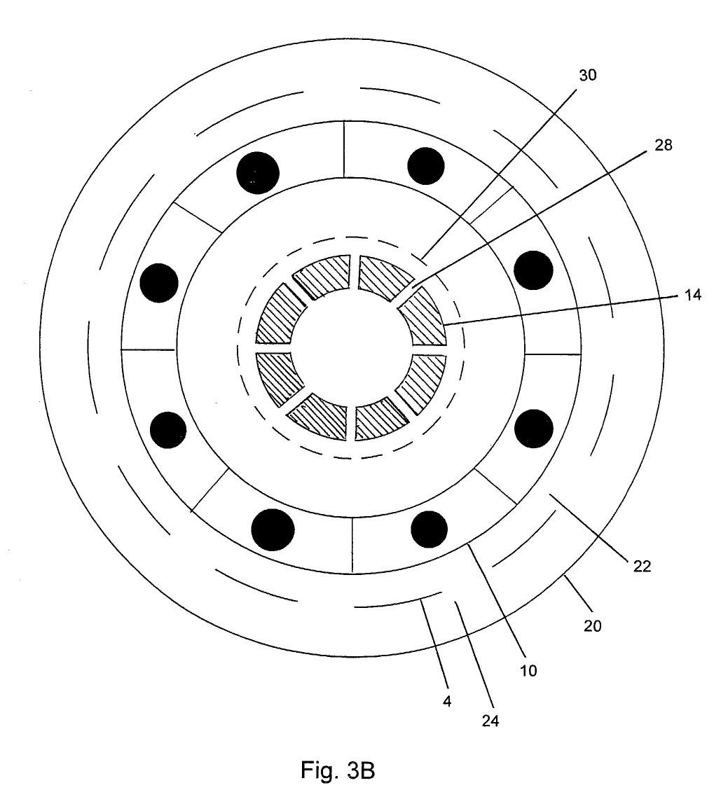

Three-dimensional embodiments are shown in FIGS. 2a, 2b, 3a, and 3b, and are described below in Examples 3 and 4.

### EXAMPLE 1

Reagents and Supplies

The L-2 cells were supplied by Ulla Wewer (Copenhagen, Denmark). The antibodies to basement membrane proteins were obtained from various sources. The heparin sulfate proteoglycan (perlecan) antibody C17, the s-laminin (62 chain) antibody C4, and the type IV collagen antibody were obtained from the University of Iowa Developmental Studies Hybridoma Bank (Iowa City, Iowa), and are described in J. R. Sanes et al., “The Basal Lamina of the Neuromuscular Junction,” Cold Spring Harbor Symposia on Quantitative Biology XLVIII, pp. 667-678 (1983); C. F. Eldridge et al., “Basal Lamina-associated Heparan Sulphate Proteoglycan in the Rat PNS: Characterization and Localization Using Monoclonal Antibodies,” J. Neurocytol., vol. 15, pp. 37-51 (1986); and Foellmer et al., “Methods in Laboratory Investigation. Monoclonal Antibodies to Type IV Collagen: Probes for the Study of Structure and Function of Basement Membranes,” Lab. Invest., vol. 48, pp. 639-649 (1983). A polyclonal antiserum directed against laminin-1 was developed at the Louisiana State University Health Sciences Center (Shreveport, La.), and recognizes all three polypeptide chains in laminin-1, including alpha 1, beta 1, and gamma 1. See Routh et al., “Troglitazone Suppresses the Secretion of Type I Collagen by Mesangial Cells in Vitro,” Kidney International, vol. 61, pp. 1365-1376 (2002). The monoclonal antibody 2D6 with immunoreactivity against a basement membrane proteoglycan has been previously described in K. J. McCarthy et al., “Immunological Characterization of a Basement Membrane-specific Chondroitin Sulfate Proteoglycan,” J. Cell Biol., vol. 109, pp. 3187-3198 (1989). Secondary antisera (Cy® 2-conjugated goat anti-mouse IgG and Cy® 3-conjugated donkey anti-rabbit IgG) were obtained from Jackson ImmunoResearch Laboratories, Inc. (West Grove, Pa.).

Dialysis membranes with a molecular weight exclusion of 12-14,000 Da (MWCO 1214,000) were purchased from Spectrum Medical Industries, Inc. (Houston, Tex.). Silicon rubber O-rings were purchased from Small Parts, Inc. (Miami Lakes, Fla.). A 24-well plate was purchased from Becton Dickinson (Franklin Lakes, N.J.).

### EXAMPLE 2

Construction of the Two-Dimensional CAC Prototype

The two-dimensional CAC prototype was made using a 24-well plate with an internal diameter of 1.58 cm (0.625 in). An autoclaved dialysis membrane (MWCO 12-14,000 Da) was used as limiting membrane 4 to divide the culture medium well into two chambers. This membrane allowed nutrients and growth factors below 12,000 Da to diffuse freely across the membrane. The placement of limiting membrane 4 above the cells limited the vertical distance the secreted cellular products could move to less than about 1 mm. A silicon rubber O-ring was used as stabilizer 12.

Efficacy of the CAC Apparatus

L-2 cells were maintained as previously described in D. J. Wassenhove-McCarthy et al., “Molecular Characterization of a Novel Basement Membrane-associated Proteoglycan, Leprecan,” J. Biol. Chem., vol. 274, pp. 25004-25017 (1999). The cells were cultured in Dulbeco's Modified Eagles Medium (“DMEM,” JRH Biosciences, Lenexa, Kans.), a culture medium with nutrients and growth factors, and containing L-glutamine supplemented with 10% fetal bovine serum (Hyclone, Provo, Utah) and 20 ml/l of penicillin-streptomycin (5000 IU/ml and 5000 μg/ml respectively). The cells were plated at low densities in each of the 24-well plates, and incubated at 37° C. with 10% CO₂ for 24 hr until 80% confluency. Preliminary experiments indicated that the cells died if the limiting membrane was applied at the time of plating. (Data not shown). After the cells reached 80% confluency, the autoclaved limiting membrane (MWCO 12-14,000; 1.58 cm (0.625 in) diameter) was wetted in DMEM and then placed on top of the cells in each well. A silicon O-ring was then placed on top of each the membrane, and 1.5 ml of DMEM added to the well. After 24 to 72 hr, the membrane and O-ring from each well were removed. The cells and any developed matrix were washed 2× for 3 min with phosphate buffered saline (“PBS”), and fixed with 4% buffered formalin, as previously described in L. G. Luna, Manual of Histologic Staining Methods of the Armed Forces Institute of Pathology (New York, McGraw-Hill, 1968). Cells of several wells were fixed at each of 24 hr, 48 hr, and 72 hr of culturing.

Once fixation was complete, the L-2 cells and matrix were washed 3× for 10 min with phosphate buffered saline (“PBS”), treated for 20 min with 1% bovine serum albumin (BSA) in PBS, and washed again 3× for 10 min with PBS. The BSA prevented nonspecific binding of the antibody probes to the cells by saturating the surface of the cells with the non-specific protein BSA. Antibodies to the extra cellular matrix proteins (e.g., heparin sulfate proteoglycan (perlecan) antibody C17, s-laminin (β2 chain) antibody C4, type IV collagen antibody, polyclonal antiserum and monoclonal antibody 2D6) were then applied directly to the L-2 cells and matrix. After 45 min, the L-2 cells and matrix were again washed 3× for 10 min with PBS.

After washing, the Cy®2-conjugate secondary antibody was applied to the cell-matrix complex for 45 min. However, before applying, the Cy® 2-conjugate secondary antibody was diluted in PBS to a concentration of 1:100 and centrifuged at 12,000 g for 3 min to remove fluorescent particulates.

Finally, the cell-matrix complex was washed 3×for 10 min with PBS and imaged directly within the wells, so as not to disturb the three-dimensional organization of the assembled ECMs. Digital images were acquired using an Olympus IX-70 microscope (Olympus America, Inc., Melville, N.Y.) equipped with epifluorescent illumination optics and appropriate filters. The microscope was interfaced to a SenSys camera (Roper Industries, Tucson, Ariz.) whose signal was ported to a PowerMacintosh 9600 hosting the imaging acquisition/analysis software IPLab spectrum (Scanalytics, Fairfax, Va.). (Data not shown).

When the diffusion of secreted products from L-2 cells was limited by the limiting membrane 4, a robust matrix containing laminin-1 chain was assembled within 24 hr, and continued to be formed for 48 to 72 hr. In contrast, L-2 cells grown for 24 hr without a limiting membrane 4 secreted laminin, but did not develop an organized matrix. (Data not shown). Using antibodies, the matrix formed using the CAC was found to contain basement membrane chondroitin sulfate proteoglycan (BM-CSPG) and basement membrane heparan sulfate proteoglycan (BM-HSPG), but not s-laminin. (Data not shown). The matrix also stained for type IV collagen, perlecan and BM-CSPG. The presence of these proteins indicated development of a true pericellular matrix with a composition similar to that seen in an in vivo matrix. The L-2 cells assembled a BL matrix that contained at least one isoform of several common BL-associated molecules. Interestingly, the pericellular matrices immunostained with the monoclonal antibody C17 directed against the s-laminin (βchain) were negative for L-2 cell cultures under both growth conditions. The data shows that L-2 cells do not make s-laminin, and thus is a good negative control. (Data not shown). Thus, using the CAC, L-2 cells in culture formed a robust natural BL matrix without the presence of fibroblasts or molecules secreted by any other cells.

### EXAMPLE 3

Three Dimensional CAC Apparatus

FIGS. 2A and 2B illustrate one embodiment of the CAC apparatus used to grow an extracellular matrix in three-dimensions. FIG. 2A illustrates a perspective view of part of the three-dimensional CAC apparatus. As shown in FIG. 2A, the limiting membrane 4 was a tubular dialysis membrane (semipermeable) (MWCO of 12-14,000 Da; 5 mm diameter), placed over a solid tubular core 14 (4 mm diameter) such that there was a distance of about 1 mm between the core 14 and the membrane 4. In this embodiment, tubular core 14 can be hollow or solid. The size of the chamber is easily adjusted by changing the diameter of either tubular dialysis membrane 4 or tubular core 14. Both ends of tubular dialysis membrane 4 were sealed, in this case, using a top end-cap 16 and a bottom end-cap 18. The entire structure was then placed in a culture tube 20 (not shown in FIG. 2A). FIG. 2B illustrates a cross-sectional view of the complete CAC apparatus. Once placed in culture tube 20, two chambers for fluid were formed: an inner chamber 22 between tubular core 14 and the tubular dialysis membrane 4; and an outer chamber 24 between tubular dialysis membrane 4 and the walls of culture tube 20. The cells 10 and matrix were grown in inner chamber 22. All components of the apparatus were made from a bio-compatible, nondegradable material such as polycarbonate (Lexan®) or polytetrafluoretheylene (“PTFE”).

To fill inner chamber 22 with cells, two ports 26 were made using narrow bore tubing (0.5 mm diameter). The tubing was inserted into holes drilled into one of the end-caps. As long as ports 26 were in the top end-cap 16, closure of ports 26 was not necessary. To help suspend the cells in three-dimensions, a neutral solution (1 mg/ml) of type I collagen was mixed with the cells (in this case, 1×10⁷ smooth muscle cells from the rat aorta), and injected into inner chamber 22 of the apparatus. The collagen was polymerized by incubating part of the apparatus at 37° C. for 30 min without the presence of CO₂ The unit can be incubated without immersion in a fluid for the polymerization stage. However, better heat transfer was noticed when the unit was incubated in a sterile, isotonic solution of phosphate buffered saline. A critical factor is that the unit be incubated in the absence of CO₂ which inhibits collagen polymerization. After polymerization, the apparatus was immersed in a 100 mL culture tube (culture medium holder) 20 containing DMEM medium, and incubated at 37° C. with 95% air, 5% CO₂. The medium was replaced every 4 days for 2 weeks.

Tubular dialysis membrane 4 allowed nutrients and growth factors below a certain size (in this case, 12,000 Da) to freely diffuse across membrane 4 to reach the growing cells 10, and limited the distance the extracellular secreted proteins could diffuse from the layer of cells 10.

After two weeks in culture, the apparatus was removed from culture tube 20, and tubular dialysis membrane 4 removed. A layer of cells 10 and a developed matrix that formed an even tube entirely encompassing the circumference of the tubular core was observed. The cells and matrix formed an elongated cylindrical structure with a central lumen. This cylinder had a wall thickness of approximately 1 mm, and a shape resembling that of a blood vessel.

The matrix tube was fixed and processed for immunohistochemistry staining as described in Example 2. The immunohistochemical staining indicated that the smooth muscle cells had assembled a pericellular matrix rich in laminin. The orientation of the smooth muscle cells was around the tube, an orientation similar to that of smooth muscle cells found naturally in the walls of blood vessels.

Thus, when the diffusion of secreted products from the smooth muscle cells was limited by the CAC apparatus, a composite matrix tube composed of laminin and collagen was formed. The organization of the laminin matrix varied considerably, from a fibrillar matrix surrounding only the periphery of the cells to a matrix that completely enveloped the cells and having the appearance of shrink wrap. Moreover, the smooth muscle cells had organized into an orientation around the circumference of the tubular core, similar to the orientation of smooth muscle cells in the walls of arteries and veins. The three-dimensional CAC apparatus thus was shown to grow the beginning of a vascular structure in vitro, a good beginning towards developing a substitute for damaged blood vessels. Other cells useful in growing three-dimensional matrices include endothelial cells.

### EXAMPLE 4

The Active Perfusion Three-Dimensional CAC

FIGS. 3A and 3B illustrate another embodiment of the three-dimensional CAC apparatus with a modified tubular core that allows fresh medium to reach the cells close to the core. This embodiment was designed to grow a thicker tubular extracellular matrix by allowing fresh medium to reach the cells growing near the tubular core 14. FIG. 3A illustrates a hollow tubular core 14 with lateral slots 28. In this embodiment, medium flows through the hollow core and out the lateral slots 28. The end-caps, as shown in FIG. 2B, were modified to permit active pumping of the culture medium by a peristaltic pump (Bio-Rad, Hercules, Calif.) through the inlet in top end-cap 16, into tubular core 14, and out through bottom end-cap 18. The medium was ducted by tubing to a central reservoir of culture medium, to be recycled back to top end-cap 16 using the peristaltic pump (not shown). In a prototype, the pumping rate for active perfusion was 0.5 ml/min initially, increased to 1 ml/min after 24 hr. A small portion of the medium flowed out of the tubular core 14 toward the cells. The recycled medium was changed every 4 days. FIG. 3B illustrates a cross-sectional schematic of this embodiment. A second dialysis membrane 30 (semipermeable) (MWCO of 12-14,000 Da) was placed between the cells and tubular core 14. The diameter of this membrane was chosen so the membrane tightly fit the outside of tubular core 14. This membrane helped to limit diffusion of large molecular components from the cells, and to minimize the disturbance of the cells 10 immediately adjacent to the core 14 by limiting turbulent flow of the fluid from the lateral slots 28.

Preliminary experiments using this embodiment indicated that cell viability (i.e., number of live cells/unit volume) was enhanced by this additional perfusion, especially in matrix tubes with thick walls. Using this active perfusion embodiment of the three-dimensional CAC, thick tubular matrices may be grown.

The complete disclosures of all references cited in this specification are hereby incorporated by reference. Also incorporated by reference is the complete disclosure of the following paper: M. Lauer et al., “In Vitro Matrix Assembly Induced by Critical Assembly Concentration,” which was accepted for publication by Journal of histochemistry and Cytochemistry in May 2002. In the event of an otherwise irreconcilable conflict, however, the present specification shall control.

We claim:

1. A method for growing cells in culture to maintain macromolecules secreted by the cells in close proximity to the cells, said method comprising the steps of:
(a) introducing into a culture medium holder growing cells and a culture medium suitable for supporting the growth of the cells;
(b) placing into the culture medium holder a limiting membrane to divide the holder into a first chamber containing the growing cells and culture medium, and a second chamber containing culture medium but essentially none of the growing cells; wherein the limiting membrane restricts diffusion of macromolecules from the first chamber, but allows diffusion of culture medium from the second chamber into the first chamber; and
(c) maintaining the culture medium holder and cells under conditions conducive to the growth of the cells; until the cells have formed a three-dimensional cellular and extracellular matrix that differs substantially from the pattern of cells that would form in an otherwise similar holder under otherwise similar conditions, but lacking the limiting membrane.

2. A method as recited in claim 1, wherein the limiting membrane restricts diffusion of macromolecules having a molecular weight greater than about 100,000 Daltons.

3. A method as recited in claim 1, wherein the cells secrete macromolecules in random directions.

4. A method as recited in claim 1, wherein the cells are selected from the group consisting of tumor cells, smooth muscle cells, neurons, endothelial cells, mesenchymal stem cells, and fibroblasts.

5. A method as recited in claim 1, wherein the cells comprise L-2 tumor cells.

6. A method as recited in claim 1, wherein the limiting membrane is placed on top of the cells in the bottom of the culture medium holder.

7. A method as recited in claim 1, wherein the limiting membrane is placed about 1 mm or closer to the cells.

8. A method as recited in claim 1, wherein the limiting membrane is placed between about 50 nm and about 1000 nm from the growing cells.

9. A method as recited in claim 1, additionally comprising the step of placing a stabilizer on top of the limiting membrane; wherein the stabilizer tends to maintain the position of the limiting membrane.

10. A method as recited in claim 1, additionally comprising the step of placing a second stabilizer below the limiting membrane; wherein the second stabilizer tends to maintain the position of the limiting membrane.

11. A method as recited in claim 1, wherein the extracellular matrix grows substantially faster than an extracellular matrix grown in an otherwise similar holder under otherwise similar conditions, but lacking the limiting membrane.

12. A cellular and extracellular matrix produced by the method of claim 1.

13. A method for growing mammalian cells in culture into a three-dimensional, tubular matrix, said method comprising the steps of:
(a) placing a tubular core centrally inside a tubular limiting membrane to form an inside chamber, wherein the limiting membrane restricts diffusion of macromolecules from the inside chamber, but allows diffusion of culture medium into the inside chamber;
(b) introducing the cells into the inside chamber;
(c) placing the tubular limiting membrane and tubular core inside a culture medium holder to form an outside chamber; and
(d) supplying culture medium to the outside chamber; so that the macromolecules secreted by the growing cells are restricted by the tubular limiting membrane, while the culture medium may traverse the tubular limiting membrane from the outer chamber to the inner chamber; and
(e) maintaining the culture medium holder and cells under conditions conducive to the growth of the cells; until the cells have formed a tubular three-dimensional cellular and extracellular matrix inside the tubular limiting membrane that differs substantially from the pattern of cells that would form in an otherwise similar holder under otherwise similar conditions, but lacking the limiting membrane.

14. A method as recited in claim 11, wherein the cells secrete macromolecules in random directions.

15. A method as recited in claim 11, wherein the tubular limiting membrane restricts diffusion of macromolecules with a molecular weight greater than about 100,000 Daltons.

16. A method as recited in claim 11, wherein the cells are selected from the group consisting of tumor cells, smooth muscle cells, neurons, endothelial cells, mesenchymal stem cells, and fibroblasts.

17. A method as recited in claim 11, wherein the cells comprise L-2 tumor cells.

18. A method as recited in claim 11, wherein the tubular limiting membrane is placed about 1 mm or closer to the cells.

19. A method as recited in claim 11, wherein the tubular core is hollow, and contains openings adapted to allow culture medium to be pumped through the layer of growing cells.

20. A cellular and extracellular matrix produced by the method of claim 11.

21. An apparatus for growing cells in culture to maintain macromolecules secreted by the cells in close proximity to the cells, said apparatus comprising:
(a) a culture medium holder; and
(b) a limiting membrane adapted to be placed into said culture medium holder to divide said culture medium holder into a first chamber with growing cells and culture medium and a second chamber with additional culture medium; wherein said limiting membrane restricts diffusion of macromolecules out of the first chamber but allows the diffusion of the culture medium.

22. An apparatus as recited in claim 21, wherein said limiting membrane restricts diffusion of macromolecules with a molecular weight greater than about 100,000 Daltons.

23. An apparatus as recited in claim 21, additionally comprising culture medium in said first and second chambers.

24. An apparatus as recited in claim 21, additionally comprising a layer of growing cells in said first chamber.

25. An apparatus as recited in claim 24, wherein the cells are selected from the group consisting of tumor cells, smooth muscle cells, neurons, endothelial cells, mesenchymal stem cells, and fibroblasts.

26. An apparatus as recited in claim 24, wherein the cells comprise L-2 tumor cells.

27. An apparatus as recited in claim 21, wherein said limiting membrane is about 1 mm or closer to the growing cells.

28. An apparatus as recited in claim 21, wherein said limiting membrane is between about 50 nm and about 1000 nm from the growing cells.

29. An apparatus as recited in claim 21, additionally comprising a stabilizer placed on top said limiting membrane, wherein said stabilizer tends to maintain the position of said limiting membrane.

30. An apparatus as recited in claim 21, additionally comprising a second stabilizer that is placed below said limiting membrane, wherein said second stabilizer tends to maintain the position of said limiting membrane.

31. An apparatus as recited in claim 21, wherein said apparatus supports the growth of an extracellular matrix at a substantially greater rate than the growth rate of otherwise identical extracellular matrices raised in an otherwise identical apparatus under otherwise identical conditions, but lacking the limiting membrane.

32. An apparatus for growing mammalian cells in culture into a three-dimensional, tubular matrix, said apparatus comprising:
(a) a culture medium holder;
(b) a tubular core centrally located inside said culture medium holder; and
(c) a tubular limiting membrane placed around said tubular core to divide said cell culture medium holder into an inside chamber for holding the cells and culture medium, and an outside chamber with culture medium but without a substantial number of the cells; wherein said tubular limiting membrane restricts diffusion of macromolecules from said inside chamber.

33. An apparatus as recited in claim 32, wherein said tubular limiting membrane restricts diffusion of macromolecules with a molecular weight greater than about 100,000 Daltons.

34. An apparatus as recited in claim 32, additionally comprising culture medium in said inside and outside chambers.

35. An apparatus as recited in claim 32, additionally comprising a layer of growing cells in said inside chamber.

36. An apparatus as recited in claim 35, wherein the cells are selected from the group consisting of tumor cells, smooth muscle cells, neurons, endothelial cells, mesenchymal stem cells, and fibroblasts.

37. An apparatus as recited in claim 35, wherein the cells comprise L-2 tumor cells.

38. An apparatus as recited in claim 32, wherein said tubular limiting membrane is about 1 mm or closer to the growing cells.

39. An apparatus as recited in claim 32, wherein said tubular core is hollow, and contains openings adapted to allow culture medium to be pumped through the layer of growing cells.
